(19) United States
(12) Patent Application Publication
(10) Pub. No.: US 2009/0286994 A1
(43) Pub. Date: Nov. 19, 2009

(54) Process for Synthesizing Nitramine Compounds

(75) Inventor: Adam Johnson, North Whales, PA (US)

Correspondence Address: JEFFREY B. OSTER, 8339 SE 57TH ST, MERCER ISLAND, WA, 98040, US

(73) Assignee: Nanomaterials Discovery Corporation

(21) Appl. No.: 12/399,972

(22) Filed: Mar. 8, 2009

Related U.S. Application Data
(60) Provisional application No. 61/035,729, filed on Mar. 11, 2008.

(51) Int. Cl. C07D 207/50

(52) U.S. Cl. 548/557

(57) ABSTRACT

There is disclosed a process for synthesizing nitrosamine compounds. Specifically, there is disclosed a process for synthesizing N-nitropyrrolidine.

### GOVERNMENT RIGHTS

The present invention was made under U.S. Army Contract DAAE 30-01-0-0800 TOSA#58. The U.S. Government has certain rights to this invention.

### TECHNICAL FIELD

The present disclosure provides a process for synthesizing nitramine compounds. Specifically, the present disclosure provides a preferred process for synthesizing N-nitropyrrolidine as a preferred nitramine.

### BACKGROUND

Nitrosamines have received much attention due to their carcinogenic and mutagenic potential. Therefore, much effort has gone into various processes to remove such compounds from mixtures or avoid their formation entirely. However, nitrosamine compounds could be a preferred starting material to synthesize nitramines, having potential utilities as an oxidizable fuel. Therefore, nitrosamines and nitramines would need to be synthesized in a cost effective and efficient manner. However, in view of the fact that human exposure through foods or smoke products should be limited, very little or any synthesis efforts have been developed. The present disclosure provides an improved synthesis process for synthesizing nitramine formulations due to their recent discovery of utility as an oxidizable fuel using nitrosamines.

Peroxytrifluoroacetic acid is a reagent for the oxidation of nitrosamines to nitramines (Emmons and Ferris, J. Ant. Chem. Soc. 76:4623, 1953). Other methods to oxidize nitrosamines include nitrolysis of dialkyl amides (Lamberton, Quart. Revs. 8:75, 1951) and Wright's chloride-catalyzed nitration of secondary amines, wherein dinitroxydiethylnitrosamine was converted to dinitroxydiethylnitramine in 32% yield with nitric acid and ammonium persulfate (Chute et al., Can. J Research, 27B:89, 1949). Brockman et al. (Can J Research, 27B:68, 1949) reported on the oxidation of nitrosamines to nitramines with hydrogen peroxide and nitric acid but this process suffered from very low yields of the nitramine obtained.

There is a need to make large quantities of nitramines to be used in fuel cells and other portable power devices. Due to the high cost of nitramines, the current synthesis methods require more abundant and less expensive starting materials in order to be able to produce nitramines to scale.

### SUMMARY

The present disclosure provides a process for synthesizing nitramines on a large scale, comprising adding a nitrosamine to a proxy acid solution to form the corresponding nitramine. However, nitrosamines are expensive and not readily commercially available due to their carcinogenic nature. Therefore the present invention provides a two-step process to synthesize nitramines comprising forming a nitrosamine by reacting an amine with a strong acid and a nitrate salt to form a nitrosamine; and oxidizing the nitrosamine (compound (A)) to form a corresponding nitramine (compound (B)) according to scheme I below.

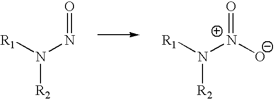

Wherein R₁ and R₂ are independently selected from the group consisting of C₁₋₆ alkyl, C₂₋₆ alkenyl, C₄₋₁₀ cycloalkyl, (R₃)₂—N—NO₂ (wherein each of the two R₃'s is independently C₁₋₆ alkyl), substituted or unsubstituted N-nitrosopyrrolidine wherein the N-nitrosopyrrolidine substitutions are at the ring 2, 3 and 4 positions and are independently, hydrogen (unsubstituted), or a C₁₋₆ alkyl, and combinations thereof. Preferably, the strong acid of the nitrosamine formation step is selected from the group consisting of sulfuric acid, trifluoroacetic acid, peroxytrifluoroacetic acid, nitric acid, formic acid, and combinations thereof. Most preferably, the strong acid is peroxytrifluoroacetic acid. Preferably, the nitrite salt is selected from the group consisting of sodium nitrite, potassium nitrite, calcium nitrite and combinations thereof. Most preferably, the nitrite salt is sodium nitrite. Preferably, the amine moiety is an amino acid, cimetidine, substituted or unsubstituted pyrrolidine, diethylamine, piperidine, piperazine and combinations thereof.

The present disclosure further provides a process for synthesizing nitramines comprising:

(a) nitrosylating an amine moiety to a nitrosamine by adding sodium nitrate into a solution of a strong acid and then adding the amine moiety to form the corresponding nitrosamine; and

(b) oxidizing the nitrosamine to its corresponding nitramine.

Preferably, the nitrosylating step uses a strong acid selected from the group consisting of HCl, sulfuric acid, nitric acid, NO₂BF₄, and combinations thereof. Preferably, the amine moiety is selected from the group consisting of N-nitrosopyrrolidine, N-nitrosodimethylamine, N-nitrosopiperidine, N,N-dinitrosopiperizene and combinations thereof.

### BRIEF DESCRIPTION OF THE DRAWINGS

FIG. 1A shows a reaction scheme for the nitration of N-nitrosamine with fuming nitric acid.

FIG. 1B shows a reaction scheme for nitration of N-nitrosamine with nitronium tetrafluoroborate.

FIG. 2 shows a reaction scheme for oxidation of N-nitrosamine with peroxytrifluoroacetic acid.

FIGS. 3A and 3B show reaction schemes for the synthesis of nitroamines from their corresponding amines.

### DETAILED DESCRIPTION

Without being bound by theory, but it appears that the superiority of using peroxytrifluoroacetic acid over other reagents for nitrosamine oxidation is due to the electrical dissymmetry of the oxygen-oxygen bond caused be the highly electronegative trifluoroacetyl radical. The present disclosure further provides a process for synthesizing nitramines comprising:

(a) nitrosylating an amine moiety to a nitrosamine by adding sodium nitrate into a solution of a strong acid and then adding the amine moiety to form the corresponding nitrosamine; and

(b) oxidizing the nitrosamine to its corresponding nitramine.

Preferably, the nitrosylating step uses a strong acid selected from the group consisting of HCl, sulfuric acid, nitric acid, and combinations thereof. Preferably, the amine moiety is selected from the group consisting of N-nitrosopyrrolidine, N-nitrosodimethylamine, N-nitrosopiperidine, N,N-dinitrosopiperazine and combinations thereof. The preferred nitrosylating agent for the conversion of a secondary amine to the corresponding nitrosamine is NaNO₂. HNO₂ (nitrous acid) acts to form the corresponding nitroso cation which is essential to the nitrosolation of the amine. The conversion to the nitrosamine is catalytic in acid.

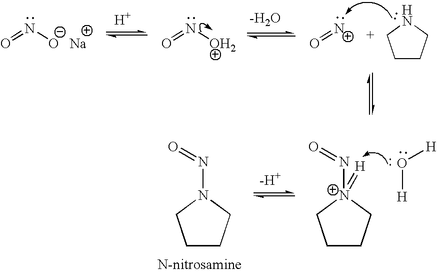

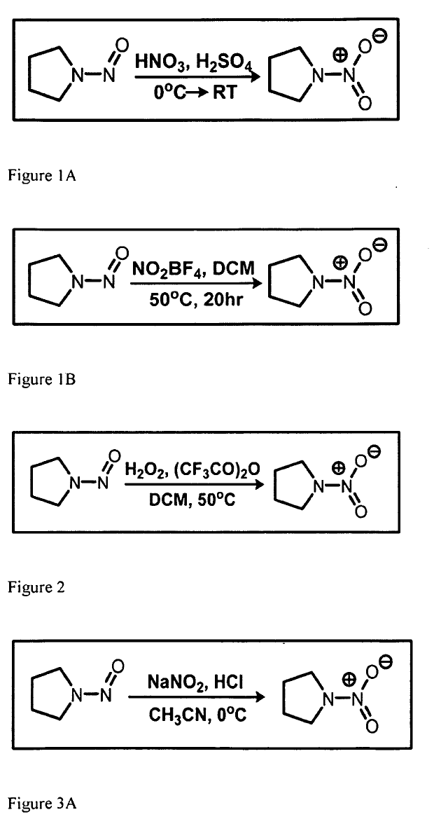

The present disclosure and process is illustrated through the synthesis of various nitroamine compounds. Specifically, N-nitropyrrolidine was attempted to be synthesized using techniques in the literature including nitrating the analogous nitrosamine in fuming nitric acid (90%) but this method gave no detectable quantity of product (Example 1 and FIG. 1A). The second attempt to synthesize N-nitropyrrolidine by nitrating nitrosamine used nitronium tetrafluoroborate instead of nitric acid (Example 2, FIG. 1B). This method did give N-nitropyrrolidine but only in very low yields.

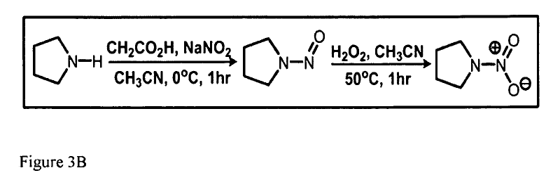

N-nitropyrrolidine was finally successfully synthesized using a third method (FIGS. 2, 3A and 3B, Examples 3 and 4) involving the oxidation of the analogous nitrosamine with a prepared peroxy acid.

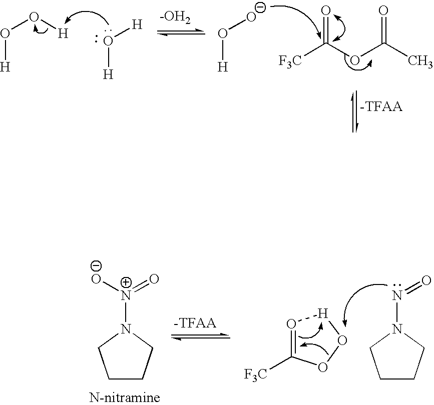

### Example 1

This example shows the attempt at nitrating an analogous nitrosamine in fuming nitric acid. The fuming nitric acid was prepared by placing 50 g. (0.495 mol) KNO₃ and 26.5 ml 98% H₂SO₄ in a 50-mL pear-shaped flask. KNO₃ undergoes a substitution reaction with the sulfuric acid to produce the KHSO₄. The reaction was distilled at ˜83° C. The potassium bisulfate remained in the flask, giving a mixture of HNO₃, H₂O and N₂O₂ in the collection flask. This product appeared orange/yellow.

Once the nitric acid had been collected, it was titrated with NaOH (6M) in a syringe to determine the percent HNO₃. Three drops of phenolthaline in EtOH was used as an indicator and the nitric acid titrated until a faint pink color was observed. The purity of the nitric acid was calculated to be ˜90% (m/m).

The fuming nitric acid was then used in the nitration reaction. 15 ml of concentrated H₂SO₄ was added to a 50-mL round bottom flask and the flask was placed in an ice bath (˜5° C.). A stir bar was placed in solution and stirred while 0.686 ml (0.0075 mol) N-nitropyrrolidine was added to the reaction. In a separate 25-mL flask, 5 ml of H₂SO₄ was mixed with 0.417 ml (0.009 mol) HNO₃. This mixture was then added slowly to the reaction on ice and the reaction was stirred at ˜5° C. for ˜1.5 hours.

Finally, the reaction was taken out of the ice-bath and let stir for ˜1 hour or longer. The reaction had a light yellow tint, and was clear with no form of precipitate visible. The work-up procedure involved pouring the reaction into 200 ml of water and neutralizing the reaction with solid K₂CO₃. The base was added rather slowly to prevent a vigorous reaction from occurring. 50 ml portions of the neutralized reaction were placed in a seperatory funnel and each portion was washed with 3×25 ml CH₂Cl₂. The organics were collected (CH₂Cl₂ is denser than water) and dried with MgSO₄ and filtered. The collected organics were taken to a rotary evaporator and the solvent removed. It should be noted that MgSO₄ is a drying agent and is used to remove water from an organic solvent.

The reaction was analyzed by GCMS. The product had a retention time of ˜4.40 minutes. However, there were no peaks were identified product (m/z=116). There was a considerable quantity of nitrosamine left (rt=˜4.90 min.). Therefore, the conditions used in this reaction were slightly wrong for this particular reaction.

### Example 2

This example illustrates a second method for nitration of a cyclic nitrosamine using nitronium tetrafluoroborate instead of nitric acid. The reaction proceeded by charging a 50-mL round bottomed-flask with 20 ml CH₂Cl₂ and adding a stir bar. 0.5 g NO₂BF₃ was then added to the reaction vessel. The reaction was stirred while 0.686 ml N-nitrosopyrrolidine was added dropwise. The reaction had a yellow tint from the nitrosamine and the NO₂BF₃ could be seen at the bottom of the reaction vessel. The reaction was stirred for ˜20 hours.

The reaction was monitored by TLC in 1 hexanes: 1 ethyl acetate and stained KMnO₄ (heat after dipping). Before the reaction was stained, it was placed under a UV lamp and each spot traced with pencil. The reaction still had a large quantity of nitrosamine after 20 hours as this spot stained yellow with KMnO₄. Furthermore, there were several spots that were unaccounted for lying below the nitrosamine and a rather bright spot at the base line. This was co-spotted with pyrrolidine and believed to be the identity of the spot. A spot above the starting material did not stain with KMnO₄ and was not identified.

Work-up for this reaction involved filtering off the excess NO₂BF₄, diluting the mother liquor with ˜50 ml CH₂Cl₂ and extracting with aqueous NaHCO₃ (3×50 ml). The organic layer was extracted once with brine (50 ml) and dried with MgSO₄. The magnesium sulfate was filtered and the organics condensed by rotary evaporation.

The reaction was analyzed by GCMS and it was evident that product had formed as there was a large peak coming off of the column at ˜6.40 min. This peak had a parent m/z=116. Although there product formed in this reaction, it was evident that the majority of the contents in the reaction mixture was still nitrosamine (˜4.80 min, m/z=100).

### Example 3

This example illustrates oxidation of N-nitrosamine with peroxytrifluoroacetic acid. In this reaction, the goal was to oxidize the nitroso group to a nitro group instead of replacing it entirely as was attempted in the two previous examples. This was done by making a peroxyacid in situ and heating the reaction for one to two hours FIG. 2). Specifically, the reaction was performed by adding 15 ml of DCM as well as a stir bar to a 50-mL reaction flask. The reaction flask was then placed in an ice-bath and trifluoroacetic anhydride (5.004 ml, 0.036 mol) was then added and the mixture stirred. 30% Hydrogen peroxide (2.429 ml, 0.028 mol) was added to the reaction flask next. When the peroxide was added to the reaction flask, the reaction boiled and it is therefore necessary to keep the ice bath at ˜0° C. In a seperatory funnel, 5 ml of DCM was added followed by N-nitrosopyrrolidine (1.83 ml, 0.02 mol). This solution was orange/yellow due to the nitrosamine. After the peroxyacid reaction had stirred for 10 min., the reaction was warmed to room temperature and the contents of the seperatory funnel were added dropwise over ˜20 min.

When the nitroamine had been added to the reaction flask, it was then connected to a condenser with a clip and sealed with grease and refluxed for ˜1.5 hrs. The reaction went from light orange to clear over that time. The reaction was worked-up by evaporating off the solvent and crystallizing the product in EtOH. White crystals were observed for pure product.

Yields for this reaction have varied from 68% to 80%.

### Example 4

This example illustrates oxidation of N-nitrosamine with peroxytrifluoroacetic acid produced in Example 3. Due to the high cost of nitrosamines research went into a method for the development of nitroamines beginning with less expensive starting materials. A method was found, in which an amine was nitrosylated to form the corresponding nitrosamine (FIG. 3A). In this reaction, the nitrosonium ion was formed by adding sodium nitrate into a 4 M solution of HCl (reaction is turns blue). The amine was then added and stirred at 0° C. for ˜1 hr. The blue color ceased to persist as the reaction progressed. This reaction was monitored by TLC(CH₂Cl₂, KMnO₄ stain).

The work-up for this reaction involved an extraction with DCM to remove excess NaCl. Once this reaction had been worked up and the volume minimized, the nitrosamine was oxidized to the nitroamine (FIG. 2). Beginning with pyrrolidine this produced N-nitropyrrolidine which was compared with a sample of the product by TLC.

By using acetic acid instead of HCl in the nitrosylation, one simply adds hydrogen peroxide in the second step in order to form the peroxyacid with the acetic acid and oxidize the nitrosamine to the nitroamine (FIG. 3B).

The final product is extracted in CH₂Cl₂ and crystallized in EtOH. In another synthesis, the amine used was piperazine and this reaction yielded the corresponding nitrosamine.

I claim:

1. A two-step process to synthesize nitramines comprising forming a nitrosamine by reacting an amine with a strong acid and a nitrate salt to form a nitrosamine; and oxidizing the nitrosamine (compound (A)) to form a corresponding nitramine (compound (B)): 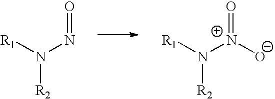
wherein R₁ and R₂ are independently selected from the group consisting of C₁₋₆ alkyl, C₂₋₆ alkenyl, C₄₋₁₀ cycloalkyl, (R₃)₂—N—NO₂ (wherein each of the two R₃'s is independently C₁₋₆ alkyl), substituted or unsubstituted N-nitrosopyrrolidine wherein the N-nitrosopyrrolidine substitutions are at the ring 2, 3 and 4 positions and are independently, hydrogen (unsubstituted), or a C₁₋₆ alkyl, and combinations thereof.

2. The two-step process to synthesize nitramines of claim 1 wherein the strong acid of the nitrosamine formation step is selected from the group consisting of sulfuric acid, trifluoroacetic acid, peroxytrifluoroacetic acid, nitric acid, formic acid, and combinations thereof.

3. The two-step process to synthesize nitramines of claim 2 wherein the strong acid is peroxytrifluoroacetic acid.

4. The two-step process to synthesize nitramines of claim 1 wherein the nitrite salt is selected from the group consisting of sodium nitrite, potassium nitrite, calcium nitrite and combinations thereof.

5. The two-step process to synthesize nitramines of claim 4 wherein the nitrite salt is sodium nitrite.

6. The two-step process to synthesize nitramines of claim 1 wherein the amine moiety is an amino acid, cimedtidine, substituted or unsubstituted pyrrolidine, piperizine, piperidine, dimethylamine and combinations thereof.

7. A process for synthesizing nitramines comprising:
(a) nitrosylating an amine moiety to a nitrosamine by adding sodium nitrate into a solution of a strong acid and then adding the amine moiety to form the corresponding nitrosamine; and
(b) oxidizing the nitrosamine to its corresponding nitramine.

8. The process for synthesizing nitramines of claim 7 wherein the nitrosylating step uses a strong acid selected from the group consisting of HCl, sulfuric acid, nitric acid, NO₂BF₄, and combinations thereof.

9. The process for synthesizing nitramines of claim 7 wherein the amine moiety is selected from the group consisting of N-nitrosopyrrolidine, N,N-dinitrosopiperizine, N-nitrosodimethylamine, N-nitrosopipiridine and combinations thereof.
